(19) United States
(12) Patent Application Publication
(10) Pub. No.: US 2026/0215778 A1
(43) Pub. Date: Jul. 30, 2026

(54) THREAD STITCHING PATTERNS FOR AN IMPLANTABLE ADJUNCT

(71) Applicant: CILAG GMBH INTERNATIONAL, ZUG (CH)

(72) Inventors: MARK ZEINER, LOVELAND, OH (US); SARAH A. SCULLY, CINCINNATI, OH (US); HEATHER STRANG, WEST CHESTER, OH (US); JOHN KIMSEY, CINCINNATI, OH (US)

(73) Assignee: CILAG GMBH INTERNATIONAL, ZUG (CH)

(21) Appl. No.: 19/183,187

(22) Filed: Apr. 18, 2025

Related U.S. Application Data
(60) Provisional application No. 63/649,648, filed on May 20, 2024.

(51) Int. Cl. A61B 17/072

(52) U.S. Cl. CPC A61B 17/07292; A61B 17/07207; A61B 2017/07271

(57) ABSTRACT

Stitching patterns for implantable adjuncts are disclosed. The stitching patterns decrease the occurrence of staple crowns pulling through the adjunct. An adjunct includes a thread passing through a full height of the implantable adjunct. The thread forms a plurality of surface segments external to the implantable adjunct. The surface segments further form a stitching pattern proximate a surface of the implantable adjunct. At least a portion of the surface segments are positioned at a first angle with respect to a longitudinal axis of the implantable adjunct. The first angle aligns the at least a portion of the surface segments such that they are non-parallel with respect to staple pocket openings on the deck of the staple cartridge.

### CROSS-REFERENCE TO RELATED APPLICATIONS

This application claims the benefit of U.S. Provisional Application No. 63/649,648, filed on May 20, 2024, the entirety of which is incorporated herein by reference.

### FIELD OF INVENTION

The present disclosure generally relates to implantable adjuncts for surgical staplers. More specifically, the present disclosure relates to designs and methods for attaching a film layer to the implantable adjuncts.

### BACKGROUND

Stapling is a crucial aspect of many surgical procedures, such as gastrointestinal, thoracic, and gynecological surgeries. A pivotal aspect of a stapling procedure is to provide proper staple formation (i.e., the legs curling around at a proper position to secure tissue within the formed staple). One issue that can impact proper staple formation is staple pull-through, i.e., the crown of the staple is pulled into the surface receiving the staple impacting the staple formation.

Certain staple cartridges used in stapling procedures may include an implantable adjunct on the deck of the cartridge or on the anvil side of the device. This implantable adjunct can be used to compensate for differences in tissue thickness. For instance, tissue may be thicker at one end of a staple cartridge than at another, yet the length of staple legs is the same for all staples in the cartridge. As such, a staple can have proper length in one section of that tissue yet be too long for another section of the tissue. The implantable adjunct is stapled to the tissue, thereby compensating for some of the thickness variation, and, in some implementations, the implantable adjunct will biodegrade over time. Accordingly, implantable adjuncts are typically comprised of a cushioned material that can be compressed during the stapling process. However, such materials are subject to staple pull through.

Since the adjunct is stapled to the tissue, the implantable adjunct must be constructed such that crowns of the staples do not pull through the adjunct—if staples pull through, then the tissue thickness is no longer compensated for. Various designs have been implemented to decrease the chance of staple pull-through. One such method is to include a film layer on the adjunct such that the adjunct comprises multiple layers, including for instance a foam or other porous material that is then laminated with a layer of film material. The multiple layers can include several layers of film and fabric located on either side of the device. However, the film material is sometimes not strong enough to prevent staple pull through. Another method of increasing pull-through strength of the object is to embed layers of higher-strength material within the adjunct. However, such methods require additional manufacturing steps and could impact the structural integrity of the implantable adjunct. There is a need for providing an implantable adjunct that provides increased resistance to staple pull through.

It is an object of the present disclosure to provide devices and methods to meet the above-stated needs. The designs can be for systems and devices for implantable adjuncts that can be adhered to stapling cartridges for use in stapling procedures that provide stitching patterns thereon that prevent staple pull through. The stitching patterns advantageously are located proximate to the staple receiving surface and prevent the staple crowns from pulling through that surface based on contact with the threaded stitching pattern. The stitching pattern can also be employed to mechanically attach one or more film layers to the implantable adjunct.

Other aspects of the present disclosure will become apparent upon reviewing the following detailed description in conjunction with the accompanying figures. Additional features or manufacturing and use steps can be included as would be appreciated and understood by a person of ordinary skill in the art.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation. It is expected that those of skill in the art can conceive of and combine elements from multiple figures to better suit the needs of the user.

FIG. 1 is a perspective view of a replaceable staple cartridge without an adjunct.

FIG. 2A is a schematic view showing a replaceable staple cartridge, an attachment material, and an implantable adjunct.

FIG. 2B is a schematic view showing the replaceable staple cartridge, attachment material, and implantable adjunct of FIG. 2A assembled.

FIG. 2C is a perspective view showing the replaceable staple cartridge and implantable adjunct of FIG. 2A assembled.

FIG. 3A is a side-view schematic of a staple cartridge being loaded into a surgical instrument, according to aspects of the present disclosure.

FIG. 3B is a schematic view of an implantable adjunct stapled to tissue, according to aspects of the present disclosure.

FIG. 4A is a perspective view of a staple cartridge deck with parallel staple pockets, according to aspects of the present disclosure.

FIG. 4B is a perspective view of a staple cartridge deck with angled staple pockets, according to aspects of the present disclosure.

FIG. 5A is a perspective view of an implantable adjunct with staples passing therethrough, the crowns of the staples showing on the surface of the adjunct, according to aspects of the present disclosure.

FIG. 5B is a perspective view of an implantable adjunct similar to that shown in FIG. 5A with the addition of a film layer, according to aspects of the present disclosure.

FIG. 6 is a perspective, exploded view of an implantable adjunct with two film layers, according to aspects of the present disclosure.

FIG. 7A is an implantable adjunct showing a stitching pattern, according to aspects of the present disclosure.

FIG. 7B is a perspective view of the implantable adjunct of FIG. 7A with staples stapled thereto, the crowns of the staples showing on the surface of the adjunct, according to aspects of the present disclosure.

FIG. 8A is a schematic of a stitching pattern supporting staple crowns positioned parallel to a longitudinal axis of the implantable adjunct, according to aspects of the present disclosure.

FIG. 8B is a schematic of a stitching pattern supporting staple crowns positioned at non-zero angles with respect to a longitudinal axis of the implantable adjunct, according to aspects of the present disclosure.

FIG. 9 is a detailed view of a staple wherein the crown compresses onto a thread of the stitching pattern, according to aspects of the present disclosure.

FIGS. 10A–10J each show variations of stitching patterns that can be used with the present designs, according to aspects of the present disclosure.

FIG. 11 is a flowchart showing an example method for assembling an implantable adjunct, according to aspects of the present disclosure.

### DETAILED DESCRIPTION

Specific examples of the present invention are now described in detail with reference to the Figures, where identical reference numbers indicate elements which are functionally similar or identical. The examples provide solutions for staple cartridge systems that include an implantable adjunct. An implantable adjunct can be used in stapling surgery to account for differing tissue thicknesses across the length of the stapling surface. For instance, a length of tissue clamped in an end effector of a surgical instrument may be thicker at one end of the staple cartridge than at the other end. However, the staple cartridge may be loaded with staples of a single length, meaning the staples may be properly sized for the thicker section of tissue, but may be too long for the thinner section of tissue. If the staples are too long, proper compression of the tissue at the staple site may not be optimal. An implantable adjunct can account for this differing tissue thickness by providing support for the thinner sections of tissue. Where the tissue is thick, the implantable adjunct can be compressed all the way down since no additional thickness is needed to account for the staple length. Where the tissue is thin, the implantable adjunct is not as compressed, meaning the adjunct provides the additional thickness needed to account for the staple length, thereby providing proper compression in that section of the tissue.

Since the adjunct is stapled to the tissue, the implantable adjunct must be constructed such that crowns of the staples do not pull through the adjunct. Staple pull through can result in the implantable adjunct not providing the necessary tissue thickness compensation and can also be dangerous for the underlying tissue. Film layers have been provided on the adjunct such that the adjunct comprises multiple layers, including for instance a foam or other porous material that is then laminated with a layer of film material. However, the film material is sometimes not strong enough to prevent staple pull through. A stitching pattern provided on the staple receiving surface of the implantable adjunct can be applied to provide sufficient strength to avoid pull through of the staple crown during a stapling operation.

The invention is not necessarily limited to the examples described, which can be varied in construction and detail. The terms “distal” and “proximal” are used throughout this description and are meant to refer to positions and directions relative to the handle of surgical instrument 200. As such, “distal” or distally” refer to a position distant to or a direction away from the handle of surgical instrument 200 (i.e., a direction toward a patient). Similarly, “proximal” or “proximally” refer to a position near or a direction towards the handle of surgical instrument 200 (i.e., toward an operator of the handle). Furthermore, the singular forms “a,” “an,” and “the” include plural referents unless the context clearly dictates otherwise. Furthermore, the use of “couple”, “coupled”, or similar phrases should not be construed as being limited to a certain number of components or a particular order of components unless the context clearly dictates otherwise.

As used herein, the terms "about" or "approximately" for any numerical values or ranges indicate a suitable dimensional tolerance that allows the part or collection of components to function for its intended purpose as described herein. More specifically, "about" or "approximately" may refer to the range of values ±20% of the recited value, e.g., “about 90%” may refer to the range of values from 71% to 109%.

The components described herein can be formed from biocompatible materials using manufacturing methods known to those of skill in the art. For example, and not limitation, the components described herein can be formed of a thermoset (e.g., the adjunct) or molded from a thermoplastic (e.g., the film or knit layers).

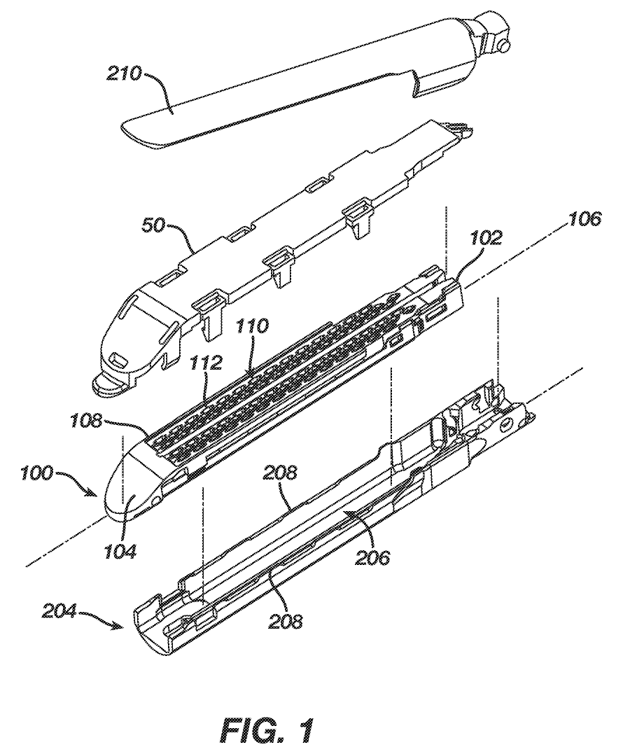

FIG. 1 provides background on how the presently described adjuncts and systems interact with a staple cartridge. FIG. 1 shows an exploded view of a staple cartridge 100 that does not include an implantable adjunct on deck 108 thereof. In these prior examples, retainer 50 can be attached to staple cartridge 100 from proximal end 102 to distal end 104 to ensure that staples within various staple pockets 110 do not fall out of openings 112 within deck 108. Retainer 50, therefore, is a static device with a function of preventing staples from falling out before staple cartridge 100 is positioned within channel 206 of first jaw frame 204 of end effector 202. Retainer 50 can be removed when staple cartridge 100 is inserted between channel rails 208 of channel 206.

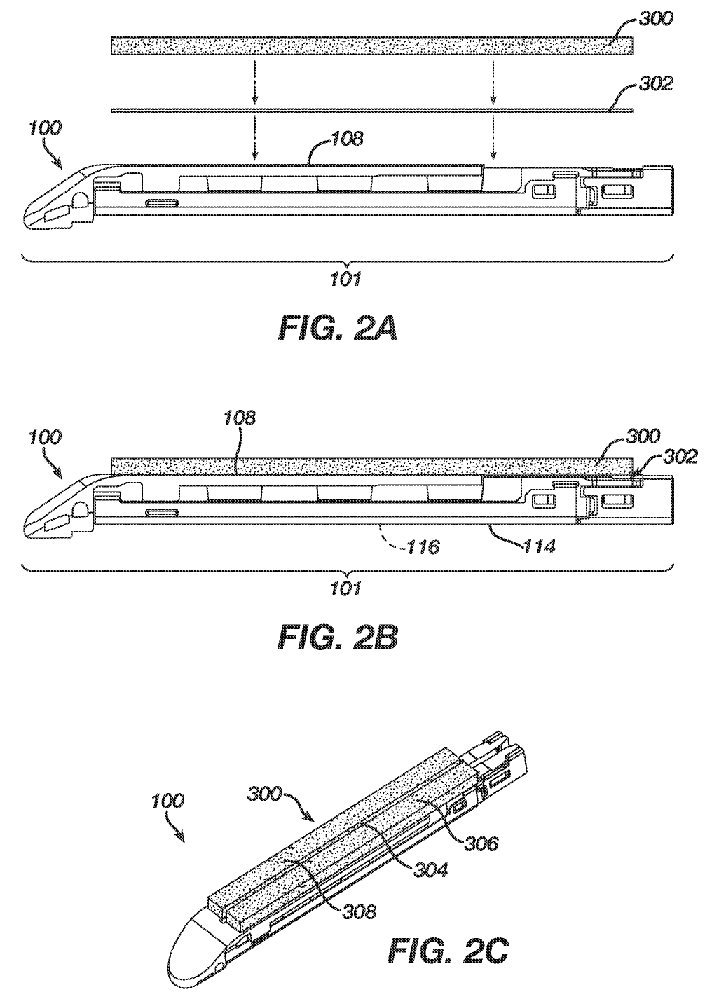

FIGS. 2A–2C illustrate staple cartridges that include an implantable adjunct 300. As seen in the exploded view of FIG. 2A, a system benefiting from the presently disclosed designs includes staple cartridge 100 (which is substantially similar to the staple cartridge shown in FIG. 1) and implantable adjunct 300 which is adhered to deck 108 that is positioned along elongate body 101 of staple cartridge 100. Adjunct 300 can be adhered to staple cartridge 100 with attachment material 302. Attachment material 302 can provide sufficient adhesion for adjunct 300 to remain adhered to deck 108 when being positioned at the treatment site, but the adhesion does not impair the ability of adjunct 300 from being detached from deck 108 when being implanted. In some instances, attachment material 302 can be an adhesive, adhesive strip, double-sided tape, and the like. The attachment material 302 can be or include a pressure sensitive adhesive such that applying pressure to the adjunct 300 causes the attachment material 302 to be compressed and increases the adhesion of the attachment material 302 to the cartridge 100 and the adjunct 300. In other words, compression of the adjunct 300 against the cartridge 100 can increase the adhesion of the adjunct 300 to the cartridge 100 via the attachment material 302. The compression of the attachment material 302 can be less than a second and greatly increases the adhesion of the adjunct 300 to the cartridge.

FIG. 2B shows adjunct 300 adhered to deck 108 via attachment material 302. FIG. 2C is a perspective view of adjunct 300 adhered to deck 108. For background, the staples of the systems described herein are fired through adjunct 300 during the stapling procedure. In some instances, adjunct 300 can include sled groove 304 along the longitudinal axis 350 of the adjunct. Sled groove 304 provides a path for a knife (not shown in figures) to traverse such that the knife does not need to cut through adjunct 300, thereby preserving the edge on the knife. When adjunct 300 includes sled groove 304, adjunct 300 can be considered to be separated into adjunct first side 306 and adjunct second side 308. In some examples, adjunct 300 can include laminated layers, such as a foam and/or porous material laminated with a mesh material, wherein the sled groove 304 is disposed in the foam and/or porous material but the mesh material remains intact. In other examples, adjunct 300 can include a film layer and/or a mesh layer and/or a knit fabric layer, as will be described in greater detail below.

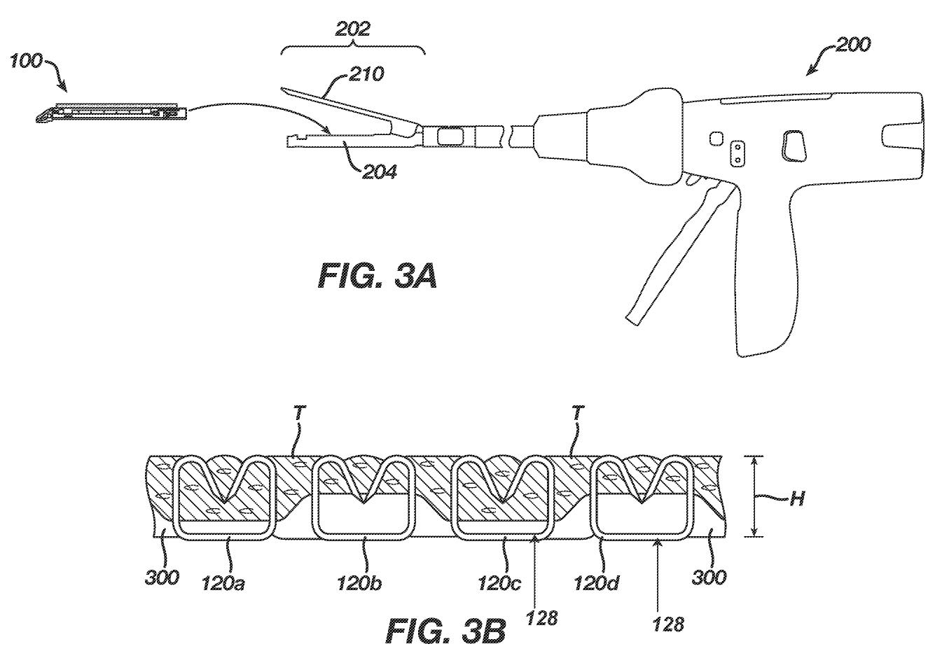

FIG. 3A is a side-view schematic of staple cartridge 100 being loaded into a surgical instrument, i.e., surgical instrument 200. Staple cartridge 100 is loaded into end effector 202 before being positioned at the treatment site. As described above, staple cartridge 100 is inserted into first jaw frame 204. Anvil 210 clamps down toward staple cartridge 100 during the stapling procedure. Once the tissue is stapled, anvil 210 opens to leave the staples and adjunct attached to the tissue. Staple cartridge 100 remains in first jaw frame 204 as surgical instrument 200 is removed from the treatment site. Although FIG. 3A shows staple cartridge 100 without a retainer attached thereto, some example retainers described herein can be configured to be inserted into first jaw frame 204 while attached to the staple cartridge 100. As is shown in FIG. 2B, cartridge 100 includes a pan 114 to support the elongate body 101, the bottom surface 117 of pan 114 being adjacent first jaw frame 204 when inserted into the first jaw frame 204 of end effector 202.

As stated above, implantable adjunct 300 can account for this differing tissue thickness by providing support for the thinner sections of tissue. Where the tissue is thick, implantable adjunct 300 can be compressed all the way down since no additional thickness is needed to account for the staple length. Where the tissue is thin, the implantable adjunct 300 is not as compressed, meaning the adjunct provides the additional thickness needed to account for the staple length, thereby providing proper compression in that section of the tissue. FIG. 3B is a schematic showing the implantable adjunct 300 stapled to tissue (T) having different thickness. The individual staples 120a,b,c,d have the same height (H), so the implantable adjunct 300 fills in the space for thinner sections of tissue (i.e., the tissue (T) shown at staples 120b and 120d). For thicker sections of tissue (i.e., the tissue (T) shown at staples 120a and 120c), the implantable adjunct 300 is more compressed as the staples do not need the additional space (i.e., height) filled in by the implantable adjunct 300. Crowns 128 of the staples 120 are shown in the figure.

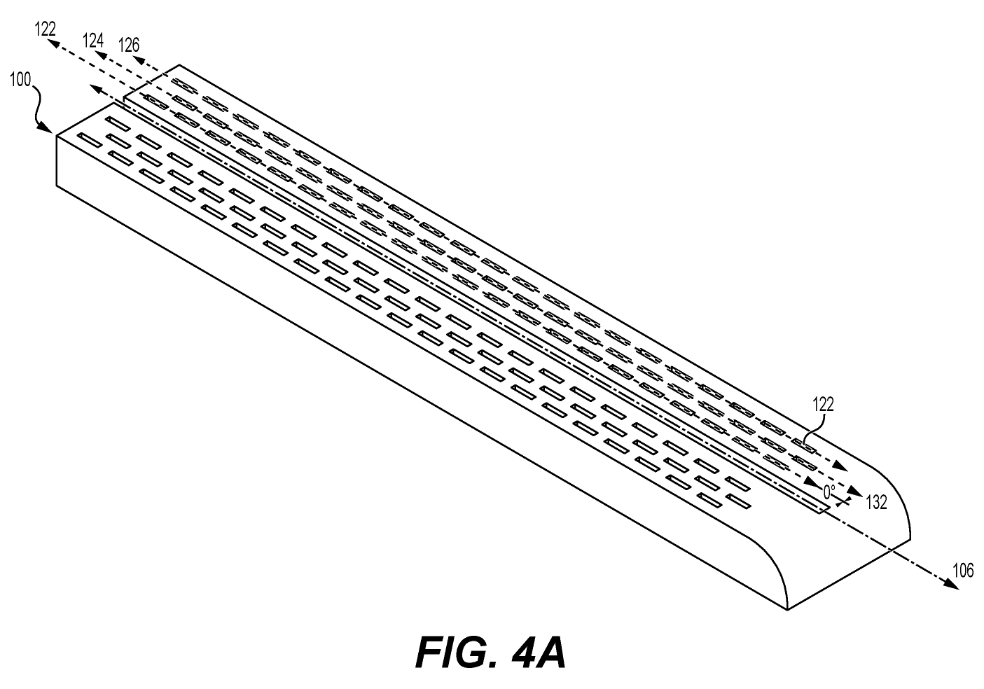

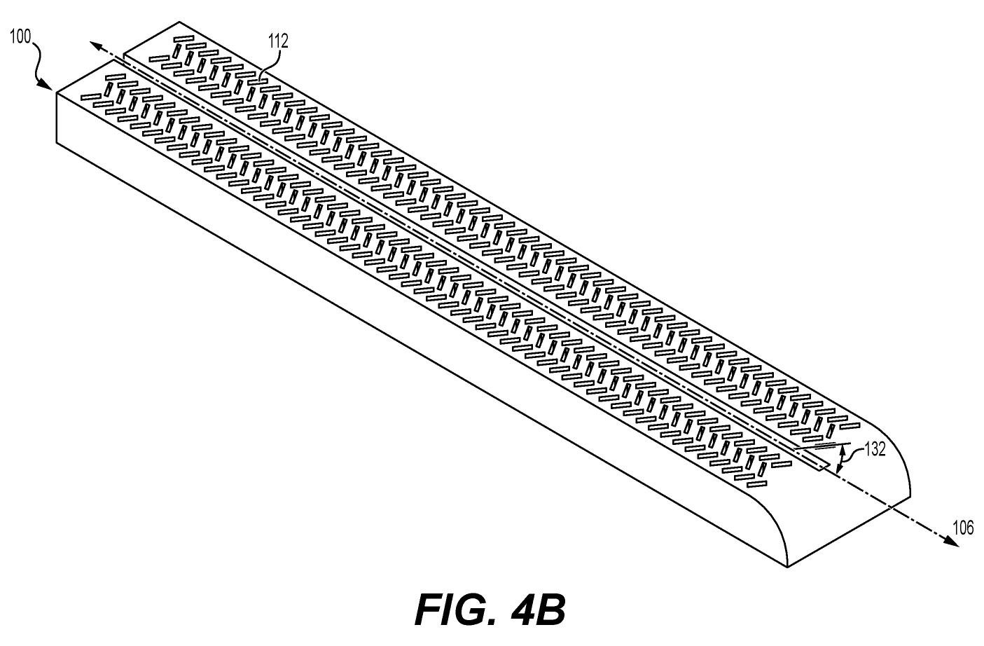

FIG. 4A is a perspective view of an exemplary staple cartridge 100 that may be employed in a system with the exemplary implantable adjuncts 300 described below to provide improved resistance to staple pull through during stapling operations. During use, implantable adjunct 300 can be detachably adhered to deck 108 of staple cartridge 100. Staple cartridge 100 includes staple pocket openings 112 in deck 108. Staple pocket openings and positioned within deck 108 and are configured for receiving a staple therethrough, such as staples 120a,b,c,d shown in FIG. 3B, as described in further detail below. In this example, staple pocket openings 112 are formed in rows and extend parallel to a longitudinal axis 106 of staple cartridge 100. For example, staple cartridge 100 includes first longitudinal row of staple pockets 122, second longitudinal row of staple pockets 124, and third longitudinal row of staple pockets 126 that each extend parallel to longitudinal axis 106, although as shown in FIG. 4A, staple cartridge 100 can include other numbers of rows of staple pocket openings 112 that extend parallel to longitudinal axis 106. In this configuration, each of staple pocket openings 112 has a staple pocket angle 132 of zero degrees with respect to longitudinal axis 106 of staple cartridge 100, although staple pocket openings 112 may be positioned at other staple pocket angles as described in the examples below.

FIG. 4B is a perspective view of another exemplary staple cartridge 100 that may be employed in a system with the exemplary implantable adjuncts 300 described below to provide improved resistance to staple pull through during stapling operations. During use, implantable adjunct 300 can be detachably adhered to deck 108 of staple cartridge 100. In this example, staple cartridge 100 includes staple pocket openings 112 in deck 108 that have a staple pocket angle 132 that is oblique with respect to longitudinal axis 106. As shown in FIG. 4B, staple pocket openings can be arranged with different staple pocket angles 132 that are oblique with respect to longitudinal axis 106.

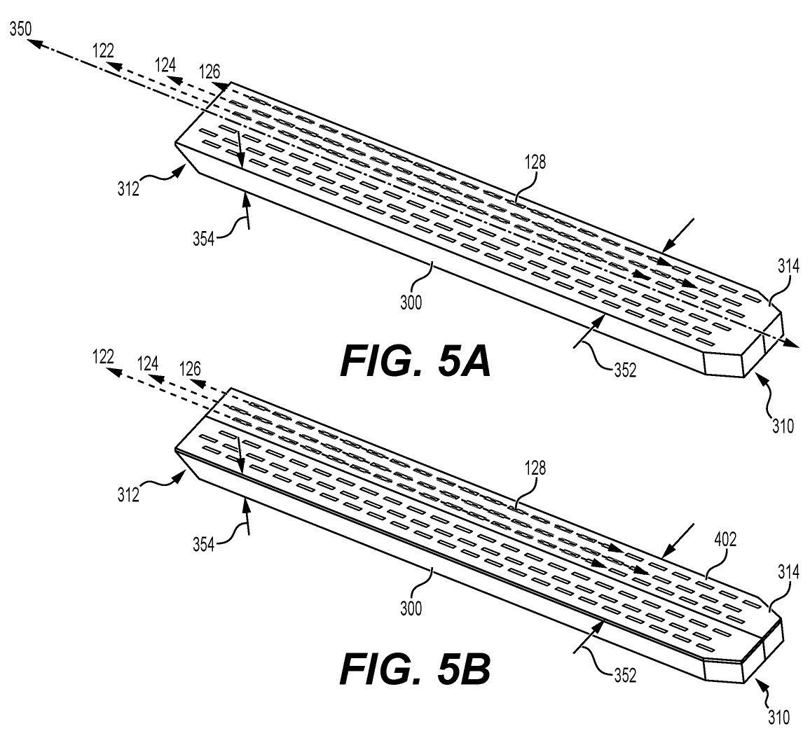

FIG. 5A is a perspective view of an exemplary implantable adjunct 300 with staples, such as staples 120a,b,c,d shown in FIG. 3B, passing therethrough. FIG. 5A illustrates the insertion of the staples using staple cartridge 100 as shown in FIG. 4A.

Implantable adjunct 300 extends along a length between a proximal end 310 and a distal end 312. Implantable adjunct 300 includes a chamfer 314 at the proximal end 310, although implantable adjunct 300 may have other configurations. The chamfer 314 can help the proximal end 310 of the implantable adjunct 300 fit between tissue stops on the proximal end of the end effector 202 (see, e.g., the tissue stops on the anvil in FIG. 3A), thereby allowing the adjunct to be positioned more proximally on within the end effector 202. In some examples, implantable adjunct 300 can be a foam and/or porous material, such as a thermoset polymer, by way of example only. In additional examples, implantable adjunct 300 can include laminated layers, such as a foam and/or porous material laminated with a mesh material. The laminated layers could include mesh, knit, film, non-woven, or other materials such as healing agents, or coagulating materials, by way of example only. Implantable adjunct 300 includes at least one layer formed of an absorbable material, as known in the art.

Implantable adjunct 300 includes a surface 316 through which staples, such as staples 120a,b,c,d shown in FIG. 3B, extend through such that crowns 128 of the staples are located on surface 316. During use, surface 316 of implantable adjunct 300 would be adhered to deck 108 of stapling cartridge 100 (as shown in FIG. 4A for example) to receive the staples. In this example, after stapling crowns 128 are located on surface 316 and extend along the lines formed by first longitudinal row of staple pockets 122, second longitudinal row of staple pockets 124, and third longitudinal row of staple pockets 126 of staple cartridge 100, which are shown in FIG. 5A to illustrate the relationship between the rows and crowns 128 after stapling. In this configuration, staple crowns 128 also extend parallel to a longitudinal axis 350 of implantable adjunct 300, which is parallel to longitudinal axis 106 of staple cartridge 100 when adhered thereto. Implantable adjunct 300 has an adjunct width 352 and an adjunct height 354.

FIG. 5B is a perspective view of an exemplary implantable adjunct 300 similar to that shown in FIG. 5A with the addition of a first film layer 402 positioned on surface 316 of implantable adjunct 300. First film layer 402 can be mechanically attached to implantable adjunct 300 using a thread, as described in further detail below. In this example, first film layer 402 would be adhered to deck 108 of staple cartridge 100 (as shown in FIG. 4A for example) and can provide a moisture barrier. In this example, first film layer 402 can be adhered to deck 108 of staple cartridge 100, for example, using attachment material 302 positioned between first film layer 402 and deck 108, as described above.

First film layer 402 can be a thin film layer formed of a thermoplastic material, although first film layer 402 can be formed of other materials. First film layer 402 can be formed in whole or in part of a biodegradable polymer, including but not limited to polymers such as polydioxanone (PDO). In some examples, first film layer 402 has a thickness of about 20 microns, although other films having other thicknesses can be employed. In this example, staple crowns 128 are located on a surface of first film layer 402, after the stapling operation is completed.

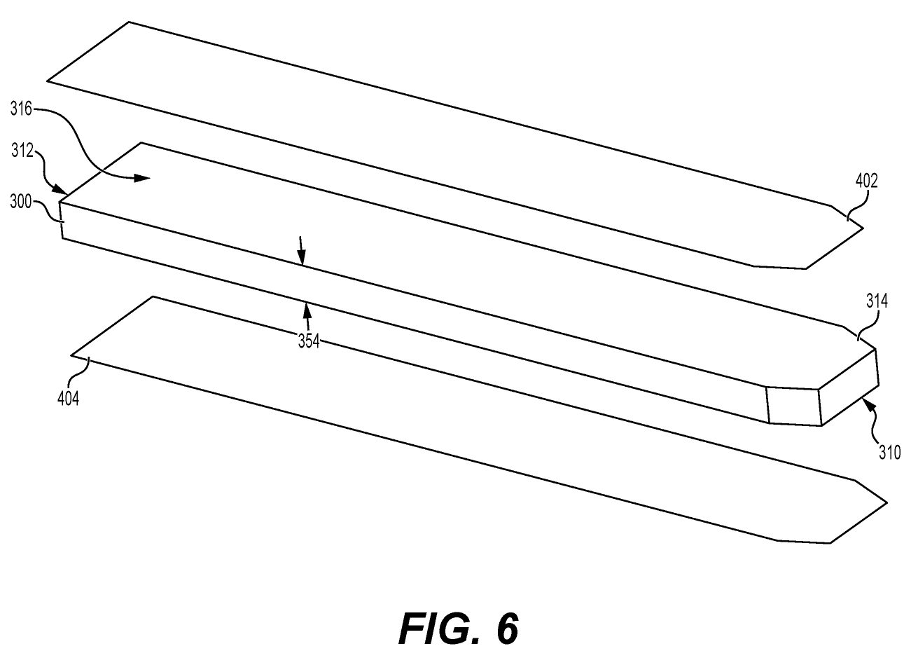

In some examples, film layers can be provided on opposing surfaces of implantable adjunct 300 and attached to implantable adjunct 300 using a thread, as described in further detail below. FIG. 6 illustrates an exploded view of an exemplary implantable adjunct 300 configured to be detachably adhered to deck 108 of staple cartridge 100. In this example, implantable adjunct 300 is shown in the exploded view with first film layer 402 and a second film layer 404 on opposing sides thereof. The film layers 402 and 404 are configured to be mechanically attached to the opposing sides of implantable adjunct 300 using a thread, as describe in further detail below. Although first film layer 402 and second film layer 404 are both shown and described, it is to be understood that in some examples, either first film layer 402 or second film layer 404 could be utilized alone with implantable adjunct 300 without including the other film layer. As described above, implantable adjunct 300 can include other laminated layers, such as a mesh material or knit fabric in addition to first film layer 402 and second film layer 404.

Second film layer 404 can be positioned to contact the surface of implantable adjunct opposite surface 316 to provide a surface that creates less friction and allows implantable adjunct 300 to glide across tissue during use. Second film layer 404 can be a thin film layer formed of a thermoplastic material, such as polydioxanone (PDO), although second film layer 404 can be formed of other materials. In some examples, second film layer 404 has a thickness of about 20 microns, although other films having other thicknesses can be employed. Second film layer 404 in some examples can be a layer that includes a discontinuous second exterior side 414, such as a mesh layer or a knit layer.

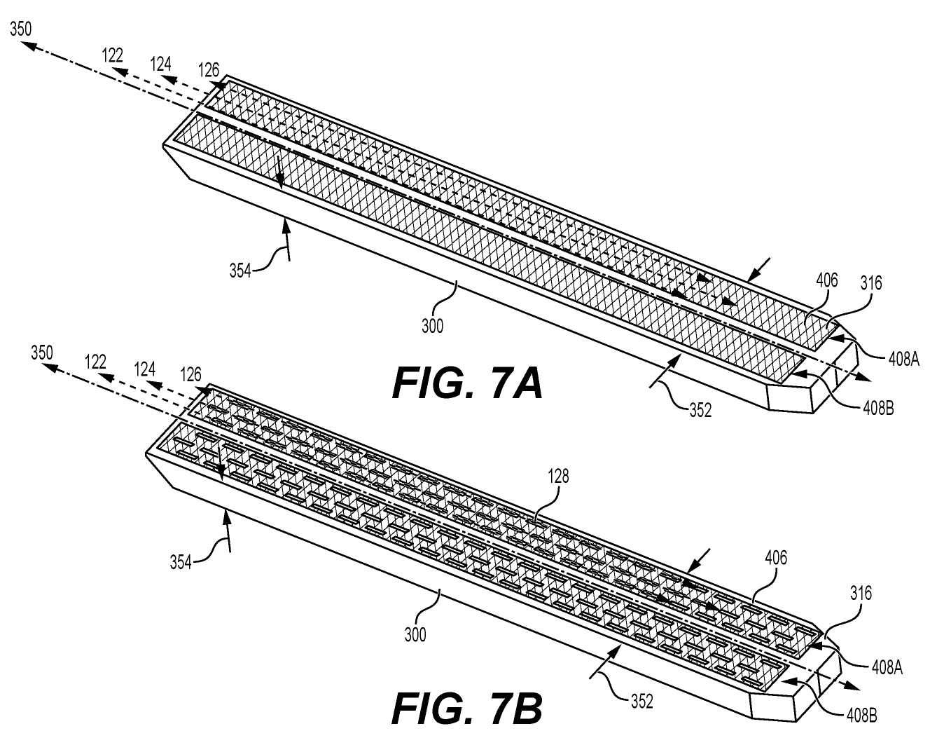

FIG. 7A shows an implantable adjunct 300 including a thread 400 forming a stitching pattern 406 proximate surface 316 of implantable adjunct 300. Various stitching techniques known in the art could be employed. For example, a lockstitch that uses upper and lower threads that entwine in the hole in the fabric which they pass through could be used. In other examples, such as when using implantable adjunct 300 shown in FIG. 5B, thread 400 can pass through and form stitching pattern 406 on a surface of first film layer 402, such that thread 400 mechanically attaches first film layer 402 to implantable adjunct 300. Thread 400 passes through the full height 354 of implantable adjunct and therefore can also be used to mechanically attach second film layer 404 (as shown in FIG. 6) to implantable adjunct 300 by passing through second film layer 404. Thread 400 can be any implantable and dissolvable suture materials known in the art. The thread can include an absorbable material, including but not limited to bioabsorbable sutures known in the art. These bioabsorbable materials for the thread can include polydioxanone (PDO), polylactic acid (PLA), polyglycolic-acid, polycaprolactone (PCA), and the like or any combination thereof.

Stitching pattern 406 can advantageously be employed to avoid staple pull through during staple operations, as described below. Implantable adjunct 300 can be employed in the system described above with respect to FIGS. 2A-2C, for example. Implantable adjunct 300 can further be employed with the exemplary staple cartridges 100, shown in FIGS. 4A and 4B.

Referring again to FIG. 7A, in this example, stitching pattern 406 includes a first stitching portion 408A and a second stitching portion 408B, although stitching pattern 406 can have other configurations. In other examples, stitching pattern 406 can extend across width 352 of implantable adjunct 300. First stitching portion 408A is positioned on surface 316 such that it is separate from and non-overlapping with second stitching portion 408B. In one example, first stitching portion 408A is separated from second stitching portion 408B by the sled groove 304 (as shown in FIG. 2C) on implantable adjunct 300.

FIG. 7A includes lines representing first longitudinal row of staple pockets 122, second longitudinal row of staple pockets 124, and third longitudinal row of staple pockets 126 of staple cartridge 100, which are shown in FIG. 7A to illustrate the relationship between the rows and stitching pattern 406. In this example, stitching pattern 406 is wider than a distance between first longitudinal row 122 and second longitudinal row 124. Stitching pattern 406 is similarly wider than a distance between second longitudinal row 124 and third longitudinal row 126.

FIG. 7B is a perspective view of implantable adjunct 320 of FIG. 7A with staples, such as staples 120a,b,c,d shown in FIG. 3B, stapled thereto. Crowns 128 are shown on surface 316 of implantable adjunct 300 with respect to stitching pattern 406. As described in further detail below, stitching pattern 406 interacts with crowns 128 to prevent staple pull through. In this example, after stapling crowns 128 are located on surface 316 and extend along the lines formed by first longitudinal row of staple pockets 122, second longitudinal row of staple pockets 124, and third longitudinal row of staple pockets 126 of staple cartridge 100, which are shown in FIG. 5A to illustrate the relationship between the rows and crowns 128 after stapling.

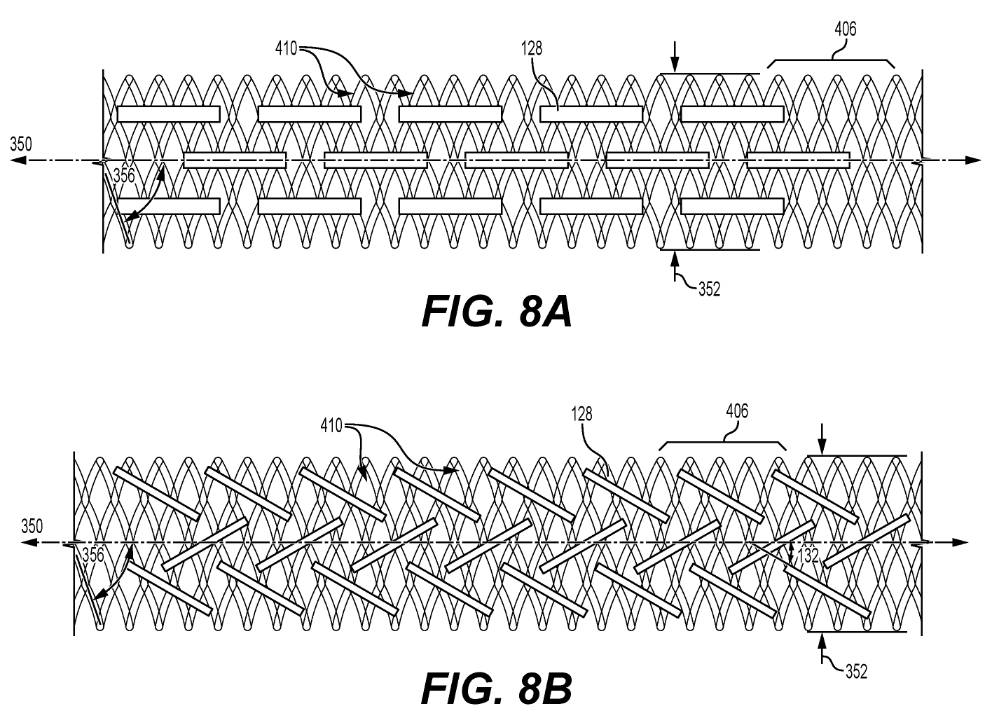

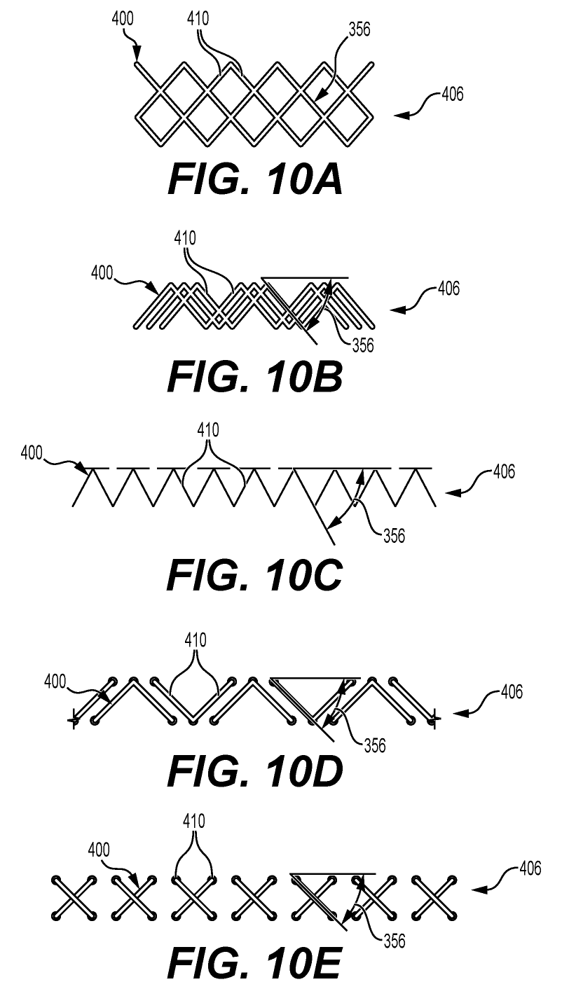

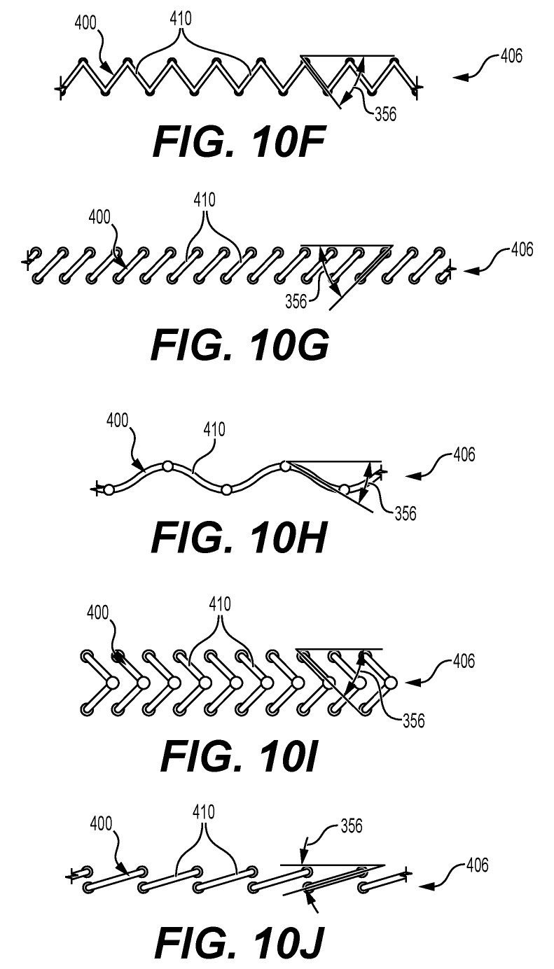

FIGS. 8A and 8B are schematics of an exemplary stitching pattern 406 employed with the staple cartridges 100 shown in FIGS. 4A and 4B, respectively. Although stitching pattern 406 is illustrated and described, it is to be understood that various stitching patterns could be employed. For example, the exemplary stitching patterns 406 shown in FIGS. 10A-10J could be employed to resist staple pull through in the same manner as described below. Further, various other stitching patterns that employ sinusoidal or otherwise rounded stitches could be employed.

Referring again to FIGS. 8A and 8B, stitching pattern 406 includes a plurality of surface segments 410 of thread 400 that are located external to implantable adjunct 300. In one example, plurality of surface segments 410 are positioned on surface 316 of implantable adjunct 300. In other examples, surface segments 410 can be positioned on a film layer, such as first film layer 402 shown in FIGS. 5A and 6, such that plurality of surface segments 410 are located proximate to surface 316. In this example, plurality of surface segments 410 provide a compressive force on first film layer 402 that mechanically attaches first film layer 402 to implantable adjunct 300. Implantable adjunct 300 can similarly have a plurality of surface segments located on the surface opposite surface 316 to attach second film layer 404 (as shown in FIG. 6).

Referring again to FIGS. 8A and 8B, stitching pattern 406 can be used to support staple crowns 128 positioned parallel to longitudinal axis 350 of implantable adjunct 300 (FIG. 8A) or staple crowns 128 positioned at non-zero angles 356 with respect to a longitudinal axis of the implantable adjunct. At least a portion of the surface segments 410 are positioned at a first angle 356 with respect to longitudinal axis 350 of implantable adjunct 300, such that they are non-parallel with respect to staple pocket openings 112 on the deck 108 of the staple cartridge 100, which determine the location of staple crowns 128 as shown in FIGS. 8A and 8B. As shown in FIG. 8B, at least a portion of surface segments 410 are positioned at a first angle 356 that is different from staple pocket angle 132, which determines the position of crowns 128.

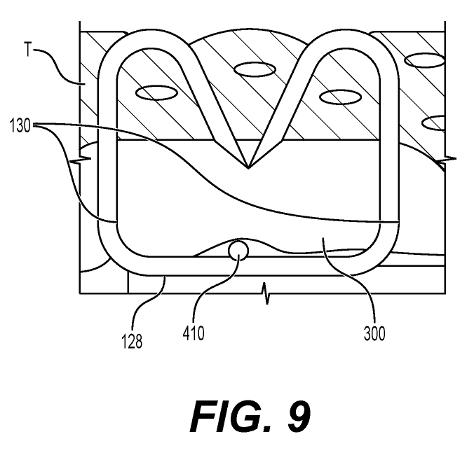

FIG. 9 illustrates a detailed view of staple 120 with crown 128 compressed onto one of the plurality of surface segments 410 of stitching pattern 406, although in other examples, crown 128 can compress a number of segments of thread 400. As shown in FIG. 9, crown 128 bridges surface segment 410, which supports crown 128 and prevents staple 120 from being pulled through implantable adjunct 300.

FIGS. 10A-10J illustrate exemplary stitching patterns 406 that could be employed on implantable adjunct 300 to resist staple pull through as described above. The stitching patterns 406 are illustrative and are not intended to be limiting as various stitching patters could be employed to prevent staple pull through. In some examples, stitching pattern 406 can formed such that all surface segments of the plurality of surface segments 410 are parallel to each other. In other examples, stitching pattern 406 can be formed such that at least two surface segments of the plurality of surface segments 410 have different angles with respect to longitudinal axis 350 of implantable adjunct 300. As discussed above, various other stitching patterns that employ sinusoidal or otherwise rounded stitches could also be employed.

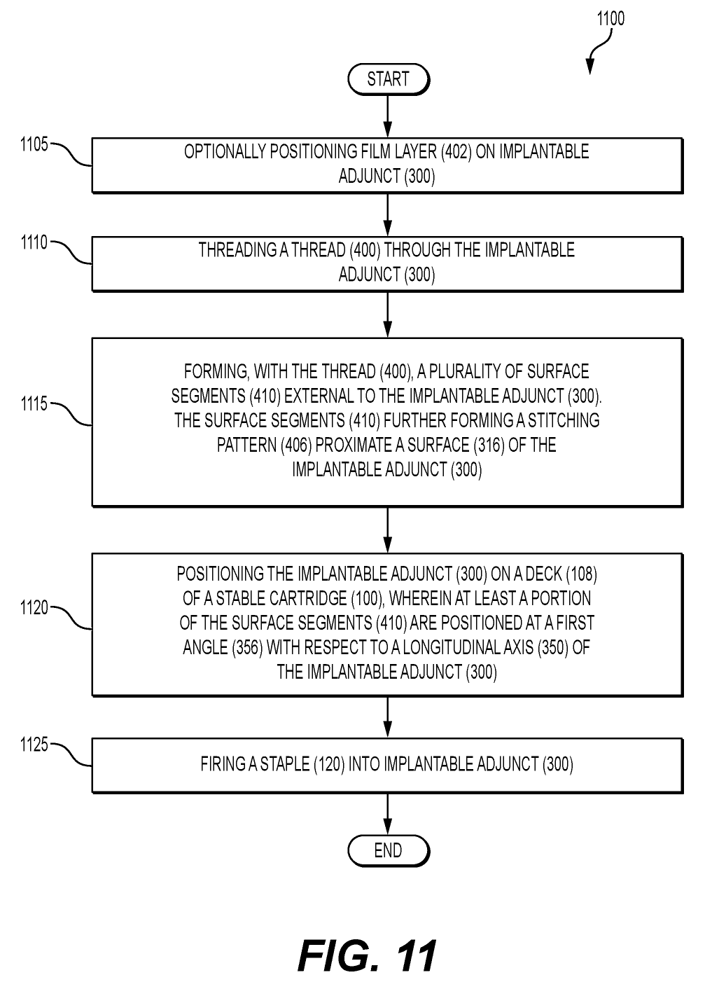

FIG. 11 is a flowchart showing an example method 1100 for assembling implantable adjunct 300 according to any of the examples set forth herein. Method 1100 will now be discussed with reference to FIGS. 1-11.

In optional step 1105, first film layer 402 is positioned on surface 316 of the implantable adjunct 300, as shown in FIG. 5B. First film layer 402 can be applied to provide a moisture barrier on surface 316 of implantable adjunct. It is to be understood that method 1100 can be performed without step 1105 in some examples.

Next, in step 1110 thread 400 is thread through implantable adjunct 300. In examples where step 1105 is performed, thread 400 is also thread through first film layer 402 and can be used to attach first film layer 402 to implantable adjunct 300. Thread 400 can be any implantable, dissolvable suture known in the art. Thread 400 can be inserted using any known sewing machines.

In step 1115, plurality of surface segments 410 are formed external to the implantable adjunct 300 using the thread 400. In some examples, surface segments 410 are formed on surface 316 of implantable adjunct. In other examples, where step 1105 is performed, surface segments 410 can be formed on first film layer 402 and provide a compressive force to attach first film layer 402 to implantable adjunct 300. Surface segments 410 are provided to form stitching pattern 406 proximate surface 316 of implantable adjunct 300. Stitching pattern 406 can have various configurations sufficient to prevent staple pull through. FIGS. 10A-10J illustrate exemplary stitching patterns 406 that may be employed, although other stapling patterns could be used to prevent staple pull through in accordance with this disclosure. In some examples, stitching pattern 406 is wider than a distance between longitudinal rows of staple openings 112, such as first longitudinal row 122 and second longitudinal row 124 shown for illustration purposes in FIG. 7A.

Next, in step 1120 implantable adjunct 300 is positioned on deck 108 of staple cartridge 100, such as the exemplary staple cartridges 100 shown in FIGS. 4A and 4B. Implantable adjunct 300 is positioned on staple cartridge 100 such that longitudinal axis 106 of staple cartridge 100 is parallel to longitudinal axis 350 of implantable adjunct 300.

Implantable adjunct 300 is positioned on staple cartridge such that at least a portion of surface segments 410 of stitching pattern 406 are positioned at first angle 356 with respect to longitudinal axis 350 of implantable adjunct 300, as shown for example in FIGS. 8A and 8B. First angle 356 aligns the at least a portion of surface segments 410 such that they are non-parallel with respect to staple pocket openings 112 on deck 108 of staple cartridge 100 when implantable adjunct 300 is attached thereto. Staple pocket openings 112 on deck 108 of staple cartridge 100 (such as shown in FIGS. 4A and 4B) are positioned at staple pocket angle 132 with respect to longitudinal axis 106 of staple cartridge 100, which is different from first angle 356. In one example, staple pocket openings 112 form longitudinal rows, such as first longitudinal row 122, second longitudinal row 124, and third longitudinal row 126, as shown in FIG. 4A by way of example, that are parallel to longitudinal axis 106 of staple cartridge 100. In this example, staple pocket angle 132 is 0°. In other examples, staple pocket openings 112 are positioned at staple pocket angle 132, which is oblique with respect to longitudinal axis 106, as shown in FIG. 4B for example.

In step 1125, staple 120 is fired into implantable adjunct 300 such that crown 128 of staple 120 bridges one or more surface segments of the plurality of surface segments 410. Accordingly, staple 120 compresses the surface segment, which prevents staple 120 from being pulled through implantable adjunct 300. Although staple 120 is described as being fired, it is to be understood that a plurality of staples could be fired in step 1125.

Examples of the present disclosure can be implemented by any of the following numbered clauses:

Clause 1: An implantable adjunct (300) configured to be detachably adhered to a deck (108) of a staple cartridge (100), the implantable adjunct (300) comprising: a thread (400) passing through a full height (354) of the implantable adjunct (300), the thread (400) forming a plurality of surface segments (410) external to the implantable adjunct (300), the surface segments (410) further forming a stitching pattern (406) proximate a surface (316) of the implantable adjunct (300), wherein at least a portion of the surface segments (410) are positioned at a first angle (356) with respect to a longitudinal axis (350) of the implantable adjunct (300), the first angle (356) aligning the at least a portion of the surface segments (410) such that they are non-parallel with respect to staple pocket openings (112) on the deck (108) of the staple cartridge (100).

Clause 2: The implantable adjunct (300) of Clause 1, further comprising a first film layer (402) contacting the surface (316) of the implantable adjunct (300), wherein the thread (400) passes through the first film layer (402).

Clause 3: The implantable adjunct (300) of Clause 2, wherein the plurality of surface segments (410) provides a compressive force on the first film layer (402), attaching the first film layer (402) to the implantable adjunct (300).

Clause 4: The implantable adjunct (300) of Clause 2 or 3, further comprising a second film layer (404) on an opposite side of the implantable adjunct (300) from the first film layer (402), wherein the thread (400) passes through the second film layer (404).

Clause 5: The implantable adjunct (300) of any one of the preceding Clauses, further comprising the staple cartridge (100), wherein: the staple pocket openings (112) on the deck (108) of the staple cartridge (100) are positioned at a second angle (132) with respect to a longitudinal axis (106) of the staple cartridge (100); the longitudinal axis (106) of the staple cartridge (100) is parallel to the longitudinal axis (350) of the implantable adjunct (300); and the second angle (132) is different than the first angle (356).

Clause 6: The implantable adjunct (300) of Clause 5, wherein the staple pocket openings (112) form a first longitudinal row (122), and wherein the second angle (132) is 0° such that the first longitudinal row (122) is parallel to the longitudinal axis (106) of the staple cartridge (100).

Clause 7: The implantable adjunct (300) of Clause 6, wherein the staple pocket openings (112) form a second longitudinal row (124), and wherein the stitching pattern (406) is wider than a distance between the first longitudinal row (122) and the second longitudinal row (124).

Clause 8: The implantable adjunct (300) of Clause 5, wherein the second angle (132) is an oblique angle.

Clause 9: The implantable adjunct (300) of any one of the preceding Clauses, wherein the stitching pattern (406) comprises a first stitching portion (408A) and a second stitching portion (408B), the first stitching portion (408A) positioned such that it is separate from and non-overlapping with the second stitching portion (408B).

Clause 10: The implantable adjunct (300) of Clause 9, wherein the first stitching portion (408A) is separated from the second stitching portion (408B) by a sled groove (304) on the implantable adjunct (300).

Clause 11: The implantable adjunct (300) of any one of the preceding Clauses, further comprising a staple (120), a crown (128) of the staple (120) is configured to bridge a first surface segment of the plurality of surface segments (410).

Clause 12: The implantable adjunct (300) of any one of the preceding Clauses, wherein the stitching pattern (406) is formed such that all surface segments of the plurality of surface segments (410) are parallel to each other.

Clause 13: The implantable adjunct (300) of any one of the preceding Clauses, wherein the stitching pattern (406) is formed such that at least two surface segments of the plurality of surface segments (410) have different angles with respect to a longitudinal axis (350) of the implantable adjunct (300).

Clause 14: A method for assembling an implantable adjunct (300), the method comprising: threading a thread (400) through the implantable adjunct (300); forming, with the thread (400), a plurality of surface segments (410) external to the implantable adjunct (300), the surface segments (410) further forming a stitching pattern (406) proximate a surface (316) of the implantable adjunct (300); and positioning the implantable adjunct (300) on a deck (108) of a staple cartridge (100), wherein at least a portion of the surface segments (410) are positioned at a first angle (356) with respect to a longitudinal axis (350) of the implantable adjunct (300), the first angle (356) aligning the at least a portion of the surface segments (410) such that they are non-parallel with respect to staple pocket openings (112) on the deck (108) of the staple cartridge (100).

Clause 15: The method of Clause 14, further comprising positioning a first film layer (402) on the surface (316) of the implantable adjunct (300) prior to threading the thread (400) through the implantable adjunct (300), wherein threading the thread (400) attaches the first film layer (402) to the surface (316).

Clause 16: The method of Clause 14, wherein: the staple pocket openings (112) on the deck (108) of the staple cartridge (100) are positioned at a second angle (132) with respect to a longitudinal axis (106) of the staple cartridge (100); the longitudinal axis (106) of the staple cartridge (100) is parallel to the longitudinal axis (350) of the implantable adjunct (300); and the second angle (132) is different than the first angle (356).

Clause 17: The method of Clause 16, wherein the staple pocket openings (112) form a first longitudinal row (122), and wherein the second angle (132) is 0° such that the first longitudinal row (122) is parallel to the longitudinal axis (106) of the staple cartridge (100).

Clause 18: The method of Clause 17, wherein the staple pocket openings (112) form a second longitudinal row (124), and wherein the stitching pattern (406) is wider than a distance between the first longitudinal row (122) and the second longitudinal row (124).

Clause 19: The method any one of Clauses 14 to 18, further comprising firing a staple (120) into the implantable adjunct (300) such that a crown (128) of the staple (120) bridges a first surface segment of the plurality of surface segments (410).

Clause 20: A system comprising: a staple cartridge (100) comprising a deck (108), the deck (108) comprising a staple pocket (110) containing a staple (120), the staple pocket (110) open at one end at a staple pocket opening (112) on the deck (108); and an implantable adjunct (300) comprising a thread (400) passing through a full height (354) of the implantable adjunct (300), the thread (400) forming a plurality of surface segments (410) external to the implantable adjunct (300), the surface segments (410) further forming a stitching pattern (406) proximate a surface (316) of the implantable adjunct (300), wherein a first surface segment of the plurality of surface segments (410) is positioned at a first angle (356) with respect to a longitudinal axis (350) of the implantable adjunct (300), the first angle (356) aligning the first surface segment such that it is non-parallel with respect to the staple pocket opening (112).

In describing example embodiments, terminology has been resorted to for the sake of clarity. As a result, not all possible combinations have been listed, and such variants are often apparent to those of skill in the art and are intended to be within the scope of the claims which follow. It is intended that each term contemplates its broadest meaning as understood by those skilled in the art and includes all technical equivalents that operate in a similar manner to accomplish a similar purpose without departing from the scope and spirit of the invention. It is also to be understood that the mention of one or more steps of a method does not preclude the presence of additional method steps or intervening method steps between those steps expressly identified. Similarly, some steps of a method can be performed in a different order than those described herein without departing from the scope of the disclosed technology.

What is claimed is:

1. An implantable adjunct (300) configured to be detachably adhered to a deck (108) of a staple cartridge (100), the implantable adjunct (300) comprising:
a thread (400) passing through a full height (354) of the implantable adjunct (300), the thread (400) forming a plurality of surface segments (410) external to the implantable adjunct (300), the surface segments (410) further forming a stitching pattern (406) proximate a surface (316) of the implantable adjunct (300),
wherein at least a portion of the surface segments (410) are positioned at a first angle (356) with respect to a longitudinal axis (350) of the implantable adjunct (300), the first angle (356) aligning the at least a portion of the surface segments (410) such that they are non-parallel with respect to staple pocket openings (112) on the deck (108) of the staple cartridge (100).

2. The implantable adjunct (300) of claim 1, further comprising a first film layer (402) contacting the surface (316) of the implantable adjunct (300), wherein the thread (400) passes through the first film layer (402).

3. The implantable adjunct (300) of claim 2, wherein the plurality of surface segments (410) provides a compressive force on the first film layer (402), attaching the first film layer (402) to the implantable adjunct (300).

4. The implantable adjunct (300) of claim 2 or 3 , further comprising a second film layer (404) on an opposite side of the implantable adjunct (300) from the first film layer (402), wherein the thread (400) passes through the second film layer (404).

5. The implantable adjunct (300) of any one of the preceding claims, further comprising the staple cartridge (100), wherein:
the staple pocket opening (112) on the deck (108) of the staple cartridge (100) are positioned at a second angle (132) with respect to a longitudinal axis (106) of the staple cartridge (100);
the longitudinal axis (106) of the staple cartridge (100) is parallel to the longitudinal axis (350) of the implantable adjunct (300); and
the second angle (132) is different than the first angle (356).

6. The implantable adjunct (300) of claim 5, wherein the staple pocket openings (112) form a first longitudinal row (122), and wherein the second angle (132) is 0° such that the first longitudinal row (122) is parallel to the longitudinal axis (106) of the staple cartridge (100).

7. The implantable adjunct (300) of claim 6, wherein the staple pocket openings (112) form a second longitudinal row (124), and wherein the stitching pattern (406) is wider than a distance between the first longitudinal row (122) and the second longitudinal row (124).

8. The implantable adjunct (300) of claim 5, wherein the second angle (132) is an oblique angle.

9. The implantable adjunct (300) of any one of the preceding claims, wherein the stitching pattern (406) comprises a first stitching portion (408A) and second stitching portion (408B), the first stitching portion (408A) positioned such that it is separate from and non-overlapping with the second stitching portion (408B), optionally wherein the first stitching portion (408A) is separated from the second stitching portion (408B) by a sled groove (304) on the implantable adjunct (300).

10. The implantable adjunct (300) of any one of the preceding claims, further comprising a staple (120), a crown (128) of the staple (120) is configured to bridge a first surface segment of the plurality of surface segments (410).

11. The implantable adjunct (300) of any one of the preceding claims, wherein the stitching pattern (406) is formed such that all surface segments of the plurality of surface segments (410) are parallel to each other.

12. The implantable adjunct (300) of any one of the preceding claims, wherein the stitching pattern (406) is formed such that at least two surface segments of the plurality of surface segments (410) have different angles with respect to a longitudinal axis (350) of the implantable adjunct (300).

13. A method for assembling an implantable adjunct (300), the method comprising:
threading a thread (400) through the implantable adjunct (300);
forming, with the thread (400), a plurality of surface segments (410) external to the implantable adjunct (300), the surface segments (410) further forming a stitching pattern (406) proximate a surface (316) of the implantable adjunct (300); and
positioning the implantable adjunct (300) on a deck (108) of a staple cartridge (100), wherein at least a portion of the surface segments (410) are positioned at a first angle (356) with respect to a longitudinal axis (350) of the implantable adjunct (300), the first angle (356) aligning the at least a portion of the surface segments (410) such that they are non-parallel with respect to staple pocket openings (112) on the deck (108) of the staple cartridge (100).

14. The method of claim 13, further comprising positioning a first film layer (402) on the surface (316) of the implantable adjunct (300) prior to threading the thread (400) through the implantable adjunct (300), wherein threading the thread (400) attaches the first film layer (402) to the surface (316).

15. The method of claim 13, wherein:
the staple pocket openings (112) on the deck (108) of the staple cartridge (100) are positioned at a second angle (132) with respect to a longitudinal axis (106) of the staple cartridge (100);
the longitudinal axis (106) of the staple cartridge (100) is parallel to the longitudinal axis (350) of the implantable adjunct (300); and
the second angle (132) is different than the first angle (356).
